(19) United States
(12) Patent Application Publication
(10) Pub. No.: US 2004/0044339 A1
(43) Pub. Date: Mar. 4, 2004

(54) Method for operating an instrument for use in high-frequency surgery, and electrosurgical device

(76) Inventors: Jurgen Beller, Gomaringen (DE); Florian Eisele, Tubingen (DE); Klaus Fischer, Nagold (DE)

Correspondence Address: Roger A Heppermann, Marshall Gerstein & Borun, 233 S Wacker Drive, Suite 6300, Sears Tower, Chicago, IL, 60606-6357, US

(21) Appl. No.: 10/432,213

(22) PCT Filed: Nov. 21, 2001
(86) PCT No.: PCT/EP01/13537

(30) Foreign Application Priority Data
Nov. 21, 2000 (DE) 100 57 585.4

(51) Int. Cl. A61B 18/18

(52) U.S. Cl. 606/34

(57) ABSTRACT

The invention relates to a method for operating an instrument (30) for use in high-frequency surgery that is connected to at least one device (10) for performing high-frequency surgery. According to the method, operational data of the device (10) is recorded at least during the first use of the instrument (30) that is connected to the device. The operational data is sent to a storage device (33), which is connected to the instrument (30) and in which the operational data is stored. The operational data is sent to a data acquisition device during a resumed or additional use and/or when testing the instrument. The device (10) is set, according to the operational data, during the first use such that the instruments (3) can be operated in the same manner as during the first use and/or the operational data can be evaluated in order to test the instrument (30).

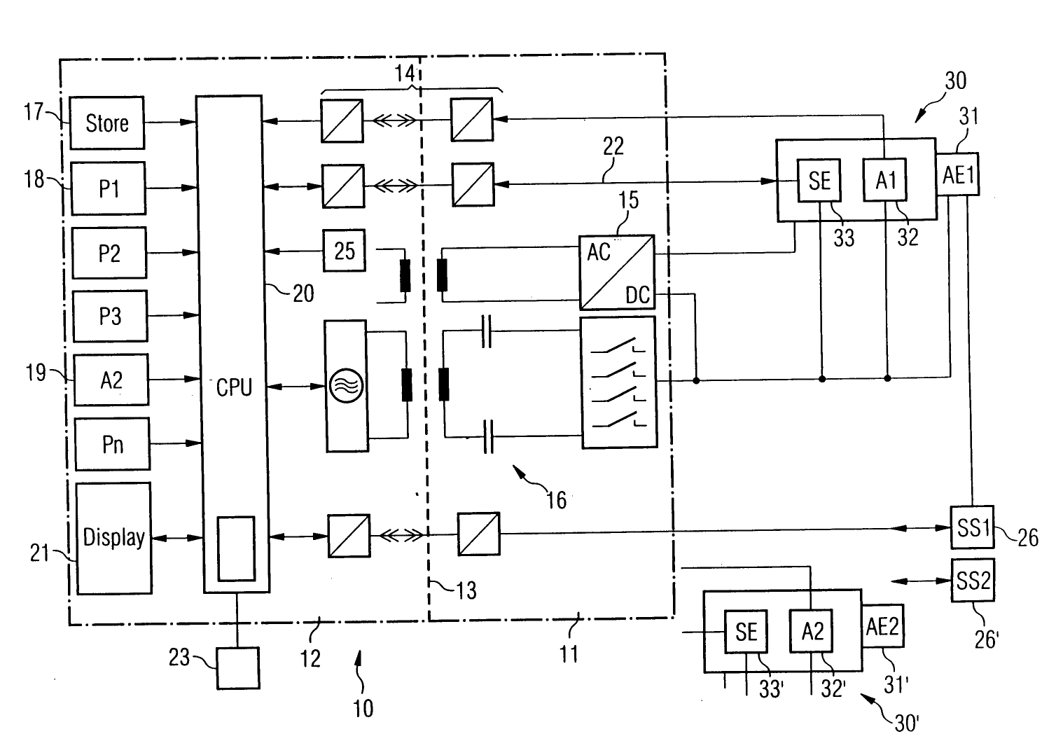

RELATED U.S. APPLICATION

This application is the U.S. national stage of corresponding PCT application No. PCT/EP01/13537 filed Nov. 21, 2001 and designating the United States, the entire disclosure of which is incorporated by reference herein.

### FIELD OF THE INVENTION

The invention relates to a method for operating an instrument for HF surgery, as well as an electrosurgical device.

### BACKGROUND OF THE INVENTION

Electrosurgical apparatus operated by high-frequency currents has become increasingly significant in recent years. In general such arrangements comprise an instrument that can be manipulated by the surgeon as well as at least one device to which the instrument is connected. The device both supplies a high-frequency electrical current and is used to control “auxiliary” functions such as the introduction of a noble gas, the application of suction to remove smoke produced during the operation, and the actions of irrigation tools or similar accessories. With electrosurgical apparatus of this kind many surgical interventions can be carried out under a great variety of conditions, both in open surgery and in (minimally invasive) endoscopic operations, where tissue is to be cut, coagulated, “glued” or treated in other ways.

On one hand such devices offer the major advantage that they can be adjusted very specifically to suit the operation being performed, even taking into account the surgeon's particular working habits. On the other hand, however, this is associated with a great disadvantage, which everyone will have noticed when programming a video recorder or adjusting a car radio: there are simply “too many possibilities for setting things, people lose their way.”

### BRIEF SUMMARY OF THE INVENTION

It is the object of the invention to provide a method for operating, a HF-surgical instrument or electrosurgical apparatus that can be employed in a simple manner, optimal for the particular application.

An essential point of the invention lies in the fact that it enables an individually specified configuration of HF-surgical systems for an HF-surgical instrument. That is, once the surgeon has decided on settings that are tailored not only to the purpose of the operation but also to his personal, individual habits, abilities and preferences, he can not only easily find them again by simply plugging “his” instrument into an available apparatus, but even more, he can immediately adopt these settings. Hence an exchange of instruments is possible with no complications, because the surgeon is immediately using the instrument with the operational data that he knows and wants to find installed. If during an operation he wishes to change the operational data, he can undertake these changes at the device in the customary manner and—if the new mode of operation seems better—adopt them for the future. That is, it is a matter of individualizing the instrument that the surgeon uses. He has a “personal” set of surgical tools, which he can always take with him.

In particular, the object stated above is achieved by a method of operating an instrument for HF surgery by way of at least one device for HF surgery, namely a method comprising the following steps:

recording the operational data for the device at least during a first period in which the instrument connected to the device is employed; in this first employment the device can be used either with a model or during an actual operation;

transmission of the operational data to a memory unit connected to the instrument, and storage of the operational data that were found to be optimal during this first employment;

transmission of the operational data to a data-acquisition unit during a new or continued period of employment and/or checking of the instrument, so that the operational data previously found to be optimal can now again be communicated to the device, and the device can be set to precisely these operational specifications;

adjustment of the device according to the operational data obtained during the first period of employment, so that the instrument can be operated in the same way as during the first employment and/or the operational data can be used to check the instrument.

The term “first employment” as used above should be understood to mean the period of employment immediately preceding a subsequent employment, i.e. not necessarily the very first period during which the instrument was employed.

The operational data should be understood to include minimally “operation” and “pause”; such operational data can of course document only when and how often the device was used, so as to provide an improved service or documentation concerning the surgeon's work. In the case of an APC device such as is described, for example, in the document WO 97/11647, the term “operational data” is to be understood as denoting the specifications for voltage, “current shape” and flow of the applied noble gas. These are parameters relevant, e.g., to a surgical procedure in the esophagus for which it is difficult to decide on the settings appropriate to the particular case; a surgeon accustomed to such a procedure learns how to adjust such parameters on the basis of experience and practice, and thus naturally keeps these settings in mind for the next operation. In such a situation it is also possible to configure the memory units of an instrument that can be used for a large number of different operations in such a way that the “programs” that the surgeon considers optimal for a particular operation can be called up.

Preferably the operational data available at a given moment are stored in response to a storage-command signal, which in particular can be input manually. Thus the surgeon can decide on the precise time for storing the operational data to which he will want to refer in future, in particular for the specific purpose of the operational step that has just been completed.

To facilitate servicing and in particular also documentation for the surgeon, it is advantageous for the operational data to include information about the duration of use, the date on which the equipment was used, and/or similar data relevant to maintenance. This enables the surgeon to record very accurately the operations performed, so that a precise, scientifically based “learning process” is made possible. Such data can also, of course, be drawn upon if questions of liability arise.

Preferably the operational data also comprise user-identification data, which can be input by the user. By this means the instrument can be individualized considerably better than is possible by a simple name plate, ensuring—if the user-identification data are suitably displayed—that instruments will not be accidentally confused with one another.

Also stored in the instrument, preferably in the factory and in such a way that alteration is impossible, are identification data that are transmitted to a device when the instrument is connected thereto, in particular so that basic values of operational data can be set in advance. These basic data are chosen such that they do not contradict the operational data determined and stored by the surgeon, i.e. do not “overwrite” the latter. In the case of a “virginal” instrument, these operational data can represent the basic settings for general operation; using them as a point of departure, the surgeon can then decide on the “optimal operation”. Then as soon as the optimal operational data have been determined and stored in the instrument or the associated memory unit, the basic data previously stored in the factory are no longer used. However, it remains possible for the surgeon, in case various trials introduce erroneous settings that lead to “chaos”, to eliminate this problem by reverting to the basic factory settings.

The object is achieved with respect to the apparatus by an electrosurgical apparatus having the following characteristics:

at least one device for electrosurgery, in particular a HF generator;

an instrument for HF electrosurgery that can be manipulated by a surgeon and, after being connected to an electrical circuit on the patient side of the device, can be used to carry out treatments of biological tissue;

an operational-data-acquisition unit to collect data regarding momentary settings that affect operation of the device and of auxiliary apparatus that may in some circumstances be used together with the device;

a memory unit connected to the instrument for the storage of the operational data, in which regard it should be noted that this memory unit can be provided both in the instrument itself and also in an auxiliary apparatus;

a bidirectional data-transfer unit, in particular a data bus for transmitting the operational data from the device to the instrument and transmitting stored data from the instrument to the device.

Preferably the device is provided with a manually actuated command element, e.g. a button-operated switch, for transmitting the momentary settings that comprise the operational data into the memory units, so that these operational data can be stored in the memory unit. Such a command element can also be implemented by a hand-operated switch on the instrument or by a pedal switch.

The memory units, depending on the size of the instrument, are disposed in the instrument itself, in a plug element by which the instrument can be connected to the device, or also in a separate component. An important consideration is that between the memory unit and the instrument there is a connection that cannot be broken or can be accessed with no possibility of error, because individualization of the instrument requires communication with the contents of the associated memory unit.

The device, for instance the HF generator, comprises a bidirectional accessory data-transfer means, e.g. a plug connector for a data bus, for connection to the auxiliary apparatus, e.g. a valve for a gas source; this should be such that operational data derived from the instrument regarding adjustment of the auxiliary apparatus, as well as operational data from the auxiliary apparatus, can be transferred for storage in the memory units. By this means even very complex arrangements of devices, which thus require considerable time and experience in order to optimize their settings, can be operated in an extremely simple manner.

In the device there are preferably provided time- and/or date-generating means (e.g., a clock), the output data from which are stored in the memory units in association with operational data, in particular with times at which the instrument is used, preferably with the simultaneous storage of associated operational parameters. Such apparatus enables optimal documentation such as is described above. Furthermore, it is possible to compare critical operational data, Such as the duration of use and operating intensities, with prespecified values and to emit a warning signal if it is desirable or even essential from the manufacturer's point of view, in order to maintain optimal function, to service the instrument or even replace it with a new one.

For the purposes of servicing and/or documentation a readout means is preferably provided, with which to read out and/or print out the data stored in the memory units. This readout means can be disposed in the device (or a separate device connected thereto) or in an entirely separate unit that can be operated independently of the HF-surgical device. In this case the user takes along a “personal” memory unit for use with a particular type of instrument.

So that user identification data can be input to the memory units, i.e. for further individualization of an instrument, within the device or in an accessory device there is provided a keyboard, an interface (for connection to a PC) or similar data-input means. With this the user can enter personal data, such as his name and in some cases also the particular use for which he has optimized the instrument (i.e., has optimized the operational data stored therein). By this means it is also possible to reproduce various operating programs which—as discussed above—have been stored and assigned (i.e., by means of identification codes) to various operational situations, in case an instrument has been optimized for a variety of such situations.

It is advantageous also to provide a memory unit that cannot be altered by the user, in particular so that instrument-specific identifying and/or operational data for the instrument can be stored before it leaves the factory. This memory can be either a ROM or a region of an EEPROM that is made inaccessible to the user, the remainder being left accessible for storage of the operational data. The data stored in this unalterable memory unit or region thereof not only allow the instrument to be individualized regarding its manufacture (batch number), but also can incorporate basic operational information that, when the instrument is used for the very first time, enable the HF-surgical device connected thereto to be adjusted or a reversion to a basic constellation of settings to be carried out.

The following exemplary embodiments will now be used to explain the invention with reference to the attached drawing

### BRIEF DESCRIPTION OF THE DRAWING

The drawing shows—highly schematically—a device 10, which in this case is designed as a HF-generator.

### DETAILED DESCRIPTION OF THE INVENTION

Within the device 10 an isolating boundary 13 separates a patient circuit 11 from an intermediate circuit 12. The device 10 further comprises a calculation/control unit 20, the central processing unit (CPU). The CPU 20 controls a HF-generator circuit 16, which is put into operation by an actuator switch 19, which for example is constructed as a pedal switch. The operational parameters are preselected by the surgeon at the device 10 by way of setting members 18 (setting members P1-Pn). Operational data and other data, such as are explained further below, can be visualized on a display 21.

To the device 10 an instrument 30 can be connected by way of a plug-in connector. In the present example the instrument 30 is described as a multifunctional instrument, which can be used for both cutting and coagulating tissue by HF-surgical means. For coagulation, from an auxiliary apparatus 26, which in the present exemplary case would be a gas supply, a noble gas is sent into the instrument 30 or an active part 31 of said instrument. In this process the gas supply or the auxiliary apparatus 26 is controlled as shown in the drawing, by way of the CPU 20 in accordance with the settings installed by the setting members 18.

In an embodiment of the invention not shown here, several such instruments 30, variously differing in construction, can be connected to the device 10.

In the instrument 30, or fixedly connected thereto, a memory unit 33 and a signal switch 32 are provided. The memory unit 33 is in communication with the CPU 20 by way of a bidirectional connection 22, as is the signal switch 32 by way of an optical coupler 14. To provide power to the memory unit 33, an instrument power supply 15 is disposed in the device 10.

When an instrument 30 is first put into operation, i.e. when it is plugged into the device 10, in the manner known per se instrument data are read into the CPU 20 by way of a read-only memory arrangement provided in the instrument 30 and programmed in the factory, or by way of a plug code or similar identifying means. As a result, the CPU 20 then adopts basic settings that enable the plugged-in instrument 30 to function at a basic level. The surgeon now uses the setting members 18 to refine these settings in a way that seems appropriate to him, having found during the operation just performed that particular setting values were optimal. As soon as these “optimal data” are available, a storage key 17 on the device 10 is actuated, whereupon the CPU 20 reads out the settings in the setting members 18 and transfers these settings through the bidirectional connection 22 to the memory unit 33 in the instrument 30, which stores these settings. During subsequent use of the instrument, i.e. at another place and/or another time, if an operation is performed that requires the same operational data, when the instrument 30 is plugged into the device 10, the stored operational data are transmitted by way of the bidirectional connection 22 to the CPU 20, which then makes all the adjustments needed to reproduce the settings of the setting members 18 that were chosen at the time of storage. Furthermore, the control commands that had been transmitted from the CPU 20 to the auxiliary apparatus 26 when the operational parameters were stored, i.e. the optimal settings, are stored simultaneously and produced again during a subsequent operation, to adjust the auxiliary apparatus 26.

When several instruments 30, 30′ are combined with a single device 10, by actuation of the signal switch 32 the CPU is informed as to which of the plugged-in instruments is being used at the moment, so that the CPU 20 adopts the settings data stored in the associated memory unit 33 or 33′ and adjusts the operational parameters accordingly (including those for the auxiliary device 26).

In addition a keyboard 23 is provided, by way of which an individualization of the instruments 30, 30′ can be undertaken for instance as follows: the surgeon who will be using the instrument 30, 30′ enters his name and/or a particular term that identifies the use for which the instrument has been optimized, by way of the keyboard 23, and by actuating the storage key 17 reads the entered data into the memory unit 33, 33′ by way of the bidirectional connection 22. When the instrument 30, 30′ is again plugged into a correspondingly constructed device 10, on the display 21 the name of the surgeon and the intended use of the instrument 30, 30′ are indicated, so that the surgeon knows exactly which one of his own instruments has just been plugged in.

In an embodiment of the invention not shown here, either the signal switch 32 is appropriately constructed or an extra signal switch is provided, so that it is possible to communicate to the CPU 20 which of several “settings programs” stored in the memory unit 33, 33′ is now to be employed. This confers a great advantage particularly when a device is to be used for different purposes during an operation, so that different optimal parameter configurations will be needed. Something of this sort can, e.g. be advantageous when different kinds of coagulation are employed, each of which has been optimized by the surgeon.

It will be evident from the above that a basic idea underlying the invention resides in the individualization of the instruments 30 that are used in combination with a device 10, such that the surgeon communicates to the device 10 and to the associated auxiliary apparatus 26 the modes of operation that have been found to be optimal, without having to make the necessary adjustments once again by hand.

Furthermore, in the device 10 a time component 25 is provided, by means of which time and date signals are communicated to the CPU 20, which by way of the bidirectional connection 22 stores these signals in the memory unit 33 in such a way that they correspond to particular modes of operation. This enables documentation of the “operating history” of each instrument 30, 30′ so that, firstly, operations that have been performed can be optimally documented and, secondly, the maintenance personnel can be provided with all the data they need in order to take whatever measures are necessary but not excessive. In this regard it is also possible to make these data available not on the display 21 provided in the device 10, but rather in a separate device that has access to the bidirectional connection 22 and also to a power supply for the memory 33, but not to the HF circuit or any other connections.

### LIST OF REFERENCE NUMERALS

10 Device

11 Patient circuit

12 Intermediate circuit

13 Isolating boundary

14 Optical coupler

15 Instrument power supply

16 HF-generator circuit

17 Storage key

18 Setting member

19 Pedal switch

20 CPU

21 Display

22 Bidirectional connection

23 Keyboard

25 Time component

26 Auxiliary apparatus

30 Instrument

31 Active part

32 Signal switch

33 Memory unit

1. A method for operating an instrument for use in high-frequency surgery with at least one device for high-frequency surgery, comprising the steps of:
acquiring operational data for the device at least during a first employment of the instrument connected to the device;
transmitting the operational data to a memory unit connected to the instrument, and storing the operational data;
transmitting the operational data to a data-acquisition unit during at least one of a period of renewed employment, of continued employment, and testing of the instrument; and
adjusting the device's settings according to the operational data obtained during the first employment, so that the instrument can be operated as it was during the first employment or so that the operational data can be evaluated for the purpose of testing the instrument.

2. The method according to claim 1, wherein the momentary operational data are stored in response to a storage-command signal.

3. The method according to claim 1, wherein the momentary operational data also comprise data associated with at least one of a source of a fluid, a suction means and a similar auxiliary apparatus.

4. The method according to claim 1, wherein the operational data comprise information as to at least one of the time period of employment, the date of employment, and service information relevant to maintenance.

5. The method according to claim 1, wherein the operational data comprise user-identification data that can be input by the user.

6. The method according to claim 1, wherein one of the preceding claims, characterized in that in the instrument identification data are stored in the instrument, preferably during manufacture and preferably unalterably, and when the instrument is connected to a device the identification these data are transferred thereto, in particular so that basic settings of operational data can be installed.

7. An electrosurgical device, comprising
at least one device for HF electrosurgery;
at least one instrument that can be manipulated by a surgeon for HF electrosurgery, and that after it has been connected to a patient circuit of the device-can be used to perform treatments of biological tissue;
an operational-data-acquisition unit to collect data related to the momentary settings for operation of the device and of any auxiliary apparatus being used together with the device;
a memory unit connected to the instrument for storage of the operational data; and
a bidirectional data-transmission means for transmission of the operational data from the device to the instrument and of stored data from the instrument to the device.

8. An electrosurgical device according to claim 7, comprising a manually actuatable command means to send the momentary settings of the operational data to the memory unit so that these data are stored in the memory unit.

9. An electrosurgical device according to claim 7, wherein the memory unit is disposed in the instrument.

10. An electrosurgical device according to claim 7, wherein the device comprises bidirectional accessory data-transmission means to connect the auxiliary apparatus in such a way that operational data originating from the instrument can be transmitted for the purpose of adjusting the auxiliary apparatus and operational data from the auxiliary apparatus can be transmitted for storage in the memory units.

11. An electrosurgical device according to claim 7, comprising time- or date-generating means, the output data from which are stored in the memory units with the times at which the instrument was used.

12. An electrosurgical device according to claim 11, comprising a readout means for the output of data stored in the memory units for the purposes of servicing or documentation.

13. An electrosurgical device according to claim 7, comprising a data-input means for entering user-identification data into the memory units.

14. An electrosurgical device according to claim 7, comprising a memory that cannot be altered by the user for the storage of instrument-specific identification data pertaining to the instrument.

15. An electrosurgical device according to claim 7, wherein the memory unit is disposed in a plug by means of which the instrument is connected to the device.

16. An electrosurgical device according to claim 7, comprising time- or date-generating means, the output data from which are stored in the memory units, with the times at which the instrument was used and with the simultaneous storage of associated operational parameters.

17. An electrosurgical device according to claim 7, comprising a memory that cannot be altered by the user for the storage of operational data pertaining to the instrument.
